(19) United States
(12) Patent Application Publication
(10) Pub. No.: US 2022/0111165 A1
(43) Pub. Date: Apr. 14, 2022

(54) MULTI-VENTILATOR FOR TREATING MULTIPLE PATIENTS TO MEET SURGE CAPACITY IN EMERGENCIES

(71) Applicant: David Myr, Jerusalem (IL)

(72) Inventor: David Myr, Jerusalem (IL)

(21) Appl. No.: 17/067,990

(22) Filed: Oct. 12, 2020

(51) Int. Cl. A61M 16/00; G16H 40/20; G16H 50/70; G16H 10/60; G16H 20/40; G16H 40/67; G16H 15/00; G16H 40/40

(52) U.S. Cl. CPC A61M 16/024; A61M 2016/0033; G16H 50/70; G16H 10/60; G16H 20/40; G16H 40/67; G16H 15/00; G16H 40/40; A61M 16/0051; A61M 2205/84; A61M 2205/52; A61M 2205/3584; A61M 2205/3306; A61M 2205/583; G16H 40/20

(57) ABSTRACT

The invention discloses a single multi-ventilator for treating multiple patients using one ventilator device. Potentially, the single disclosed multi-ventilator can be used to ventilate an entire department of patients using personalized ventilation parameters and settings.

In case of emergency, such as Covid-19, health care system may be overwhelmed by a number of patients requiring ventilator medical care to keep them alive. In such a situation, a presented ventilator solves a problem of choosing which patients will receive a ventilator treatment and which will not.

The invented ventilator provides individualized artificial ventilation care for multiple connected patients to it according to parameters determined and set by a physician.

It is important to mention that control means and main processing means will be centralized in our invention, which is another advantage of the invented ventilator.

Separate inspiration and expiration lines make the ventilator safe to use and easy to clean and to disinfect.

The medical personnel can remotely monitor all connected patients, and also set and modify different personal parameters for each patient, from a single remote on-screen patient monitor display station. In such a way, the personnel will be protected from unnecessary exposure to viruses and bacteria.

### FIELD OF THE INVENTION

This invention relates to artificial breathing assistance devices and systems, and, in particular, to the ventilator device and system for treating multiple patients in disasters and/or pandemic situations.

### REFERENCES

### US Patent Documents

U.S. Pat. No. 4,598,706 by Darowski

US patent application 20170128693 by Darowski

U.S. Pat. No. 5,664,563 by Schroeder

U.S. Pat. No. 5,901,705 by Leagre

U.S. Pat. No. 10,361,001 by Darrah

U.S. Pat. No. 6,158,430 by Pfeiffer

U.S. Pat. No. 6,474,334 by Lemer

US patent application 20190125994

US patent application 20140251322 by Miller

US patent application 20190143059 by Sanborn

### Articles

“Increasing ventilator surge capacity in disasters: ventilation of four adult-human-sized sheep on a single ventilator with a modified circuit” by Paladino

https://www.ncbi.nlm.nih.dov/pubmed/18164798

https://onlinelibrary.wiley.com/doi/pdf/10.1197/j.aem.2006.05.009 “A Single Ventilator for Multiple Simulated Patients to Meet Disaster Surge” by Neyman

### BRIEF DESCRIPTION OF THE DRAWINGS

FIG. 1 presents a general overview of the invented system;

FIG. 2 provides graphical illustration of the individualized control system;

FIG. 3 schematically depicts databases in the invented system.

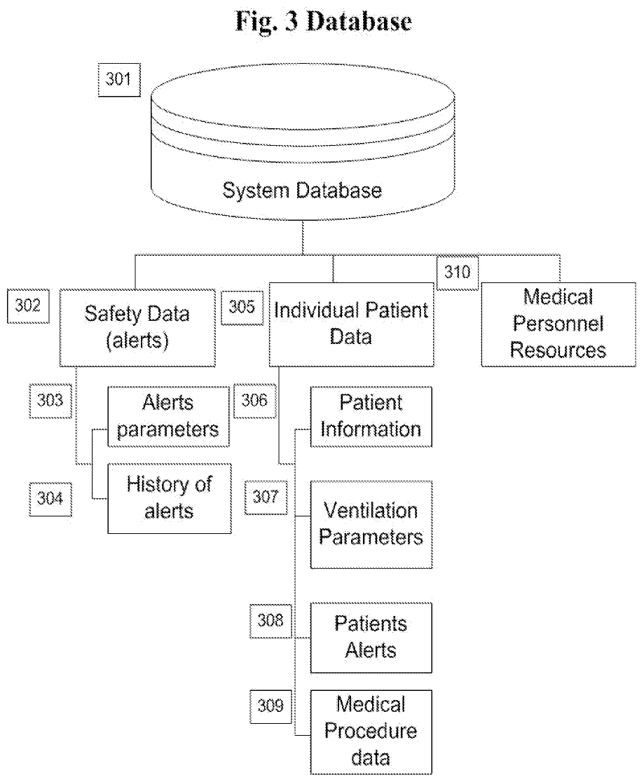

FIG. 4 shows patients' ventilation tubing and separation facilities

FIG. 5 shows Remote Wireless Operator Control Means

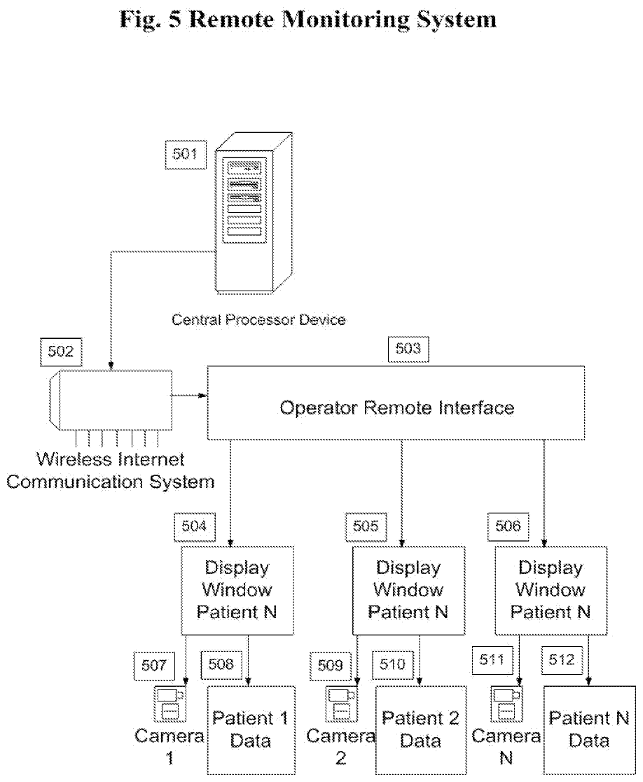

FIG. 6 graphically depicts emergency backup systems of the invented ventilator.

### NEED OF THE INVENTION

Recent health, manmade and natural disasters, such as Covid-19, have focused attention on the need to provide simultaneous care to large groups of patients.

Depending on the nature of the disaster, many hospital supplies, such as ventilators, may not be enough to support all the patients.

Clinician and public health officials have been particularly concerned about mechanical ventilator surge capacity and have suggested stock-piling ventilators and/or rationing ventilation. These possible solutions are expensive, limited by physical and human capital conditions, and sometimes unethical.

### THE STATE OF ART AND BACKGROUND OF THE INVENTION

Breathing systems generally provide oxygen to a patient, while removing carbon dioxide produced by the patient.

Patients who cannot breathe by themselves or can only partially breathe by themselves often require mechanical ventilation.

The first ventilators used on a large scale were introduced in the 1960s and were controlled and cycled by pressure. Volume controlled ventilators were introduced in the early 1970s.

There has been a great increase in the number and complexity of new ventilators.

A number of recent inventions and articles disclose modern and advanced ventilators.

U.S. Pat. No. 4,598,706 by Darowski titled “Apparatus for independent ventilation of two lungs with selective use of positive end-expiratory pressures” discloses an apparatus for controlling and dividing ventilation between lungs during their independent ventilation. The disclosed application works with two lungs of the same person. Unlike in our patent application, the apparatus disclosed by Darowski does not use personal data and personal parameters to enable it to work properly according to individualized ventilation parameters with several connected patients, each of which having different stages of disease and different needs of ventilation in terms of pressure, volume, mode of operation, etc.

US patent application 20170128693 by Darowski titled “VOLUME DIVIDER AND METHOD OF RESPIRATORY GAS Division” discloses an inspiratory gas volume divider where gas supplied by the ventilator is used to ventilate two lungs of the same person.

Unlike in our patent application, the apparatus disclosed by Darowski does not use personal data and personal parameters to enable it to work properly according to individualized ventilation parameters with several connected patients, each of which having different stages of disease and different needs of ventilation in terms of pressure, volume, mode of operation, etc.

U.S. Pat. No. 5,664,563 by Schroeder et al discloses a computer controlled pneumatic ventilator system that includes a double venturi drive and a disposable breathing circuit. The double venturi drive provides quicker completion of the exhalation phase leading to an overall improved breathing circuit. The disposable breathing circuit allows the ventilator to be utilized by multiple patients without risk of contamination. Unlike in our patent application, the disclosed ventilation system does not use personal data and personal parameters to enable it to work properly according to individualized ventilation parameters with several connected patients.

U.S. Pat. No. 5,901,705 by Leagre discloses a novel device to enable the breathing circuit to be reused on successive patients since the filter and sleeve prevent contamination from entering the breathing circuit from the patient; and prevent contamination in the breathing circuit from entering the patient. Thus, the Leagre '705 patent teaches that the replacement of the inexpensive, one-time-use filter and sleeve between patients permits the more expensive breathing circuit to be used with multiple patients.

Darrah U.S. Pat. No. 10,361,001 titled

“Autonomous critical care systems and integrated combat casualty care systems” presents a system for monitoring and treating a patient en route to a medical facility, wherein the critical care unit has a single hinge adapted to fold the critical care unit from a backpack configuration, transportable separately from a patient transport litter, into a configuration adapted to be coupled underneath the patient transport litter; at least one patient monitoring device (such as mechanical ventilator device) coupled to the critical care unit, wherein the critical care unit obtains physiological data about the patient from the at least one patient monitoring device.

U.S. Pat. No. 6,158,430 by Pfeiffer titled “Ventilator system for one or more treatment patients” discloses a ventilator system has at least one ventilator unit and a number of docking stations is described. Each docking station is adapted to receive the ventilator unit and comprises a communication interface adapted to be connected to a matching interface on the ventilator unit when the ventilator unit is docked in the docking station. The docking stations are arranged at different treatment sites, and the ventilator unit can be moved between different docking stations when necessary without interrupting the treatment of a patient connected to the ventilator unit. Unlike our invented system, Pfeiffer discloses a system where one ventilator is capable to be connected to several docking stations, but not treating several connected patients simultaneously and according to patient-individualized parameters.

U.S. Pat. No. 6,474,334 by Lemer titled “Multiplex ventilation system” presents a ventilation system including a manifold fluidly connectable at an inlet thereof to a pressurized source of a driving gas, the manifold having a plurality of branches, an electronically controlled valve fluidly connected to each branch downstream of the inlet of the manifold, a ventilator fluidly connected to each of the branches downstream of the valves, each ventilator being operative to force a driving gas into lungs of a patient, and a controller in electrical communication with the valves.

US patent application 20190125994 titled

“VENTILATORS AND SYSTEMS FOR PERFORMING AUTOMATED VENTILATION PROCEDURES” by Du; Hong-Lin presents ventilators for performing automated ventilation procedures. The automated ventilation procedure may automatically adjust ventilator parameters (e.g., PEEP) to perform a procedure such as a recruitment maneuver, PEEP titration, a recruitability assessment, or combinations thereof.

US patent application 20140251322 by Miller titled “BREATHING SYSTEMS”

discloses a modular heat exchange apparatus component that allows the base unit to be configured to avoid contact with the respiratory gases or water condensate, and hence enables the base unit to be a reusable component, with the heat exchange component being a disposable component. Therefore, such a base unit can be used safely with multiple patients by replacing the heat exchange component between patients.

US patent application 20190143059 by Sanborn titled: “Systems And Methods For Ventilation Of Patients” discloses a ventilator that includes a dashboard display identifying a patient's current ventilatory status within a global or universal ventilatory mechanics map. This dashboard display is dynamically updated with the patient's condition, and shows trends in the patient's ventilation over time.

An article by Paladino titled: “Increasing ventilator surge capacity in disasters: ventilation of four adult-human-sized sheep on a single ventilator with a modified circuit” (https://www.ncbi.nlm.nih.dov/pubmed/18164798) presents a system where adult-human-sized sheep were ventilated on a single ventilator for at least 12 hours. If the system would be transferred to humans, it would require stringent hygienic requirements, such as tubes separations, additionally, unlike in our patent application, the apparatus does not use personal data and personal parameters to enable it to work properly with several patients each of which having different stages of disease and different needs of ventilation in terms of pressure, volume, mode of operation, etc.

An article by Neyman titled “A Single Ventilator for Multiple Simulated Patients to Meet Disaster Surge”(https://onlinelibrary.wiley.com/doi/pdf/10.1197/j.aem.2006.05.009) presents a ventilator where using lung simulators, readily available plastic tubing, and ventilators, human lung simulators were added in parallel until the ventilator was ventilating the equivalent of four adults.

Unlike in our patent application, the disclosed ventilation system does not use personal data and personal parameters to enable it to work properly according to individualized ventilation parameters with several connected patients.

### DETAILED DESCRIPTION OF THE INVENTION

The disclosed single ventilator provides individualized artificial ventilation care for multiple connected patients according to medical condition of each patient.

Potentially, the single disclosed multi-ventilator can be used to ventilate an entire department of patients using personalized ventilation parameters and settings.

Using this novel ventilator, the physician can remotely choose different personal parameters for each patient on the on-screen display of the ventilator and, therefore, the invented ventilator device will provide personalized ventilator care for each connected patient.

System parameters can be set up and modified using the said on-screen display of the invented ventilator.

To separate gas flows between the patients, the invented ventilator further includes separate inspiratory paths and separate expiratory paths for each connected patient.

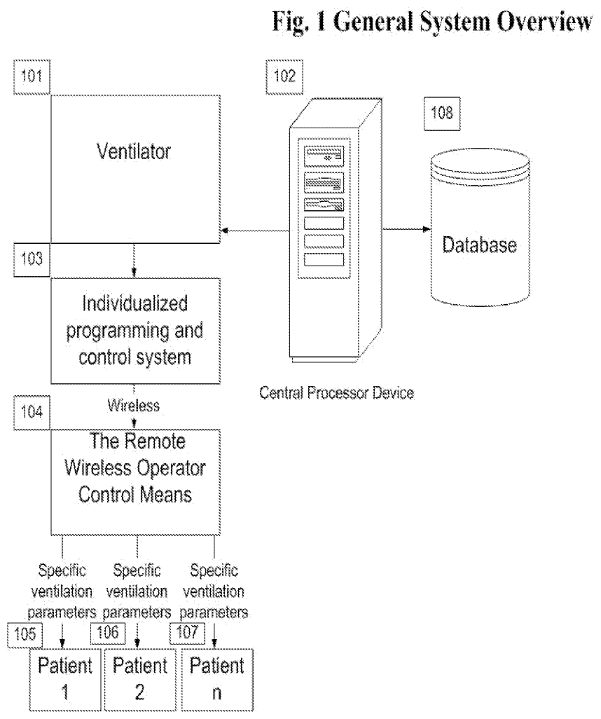

The ventilator system is graphically presented in FIG. 1.

The ventilator system comprises the following main elements:

### Ventilator Device

An invented ventilator device 101 is a single stand-alone ventilator connected to a number of patients by inspiration and expiration tubing lines for simultaneously providing individualized artificial ventilation care for two or more patients connected to the said device, such a ventilation care being provided differently to each patient according to patient-specific different ventilation care parameters, such a parameters being determined by medical personnel. The ventilator device will be equipped with the Internet connectivity means, including Wi-Fi reception and communication means, to allow the ventilator to be remotely connected and controlled.

### Central Processor Device

Such a central processor device 102 will be used for controlling individualized ventilation care parameters of each connected patient to provide such individualized ventilation care, the said central processor device will further include a database for storing and analyzing the data obtained through ventilation of the connected patients (the database functions will be detailed later in greater detail).

### Programming and Control Means (103)

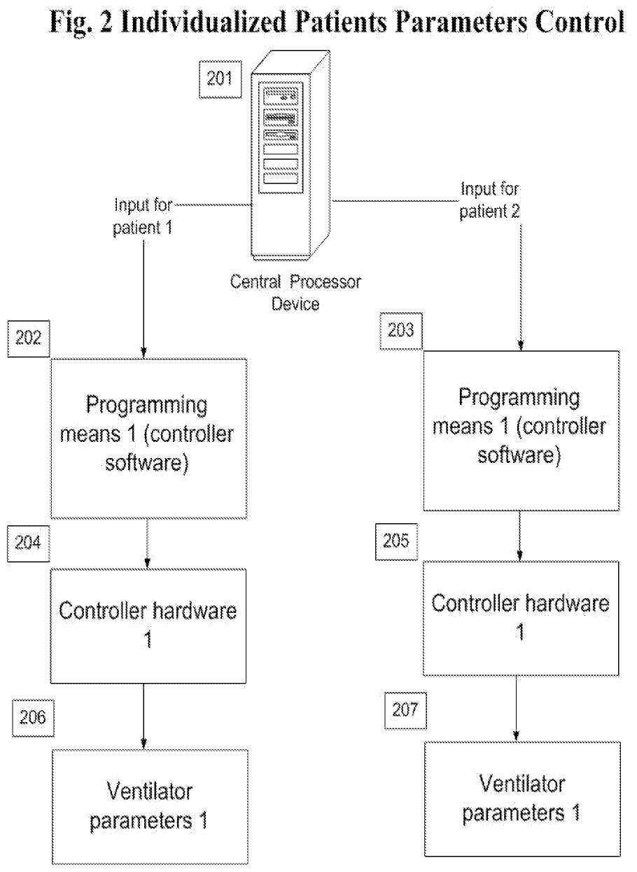

The system for setting and controlling individual patient parameters is graphically depicted in more details at FIG. 2.

From central processor device 201, an input on individual patient parameters will be provided. Programming executable instructions means, such means connected to the central processor device, and also connected to the individual inspiratory and expiratory tubes. Such programming means 202 enable the ventilator to work with individualized parameters by sending programming commands to the ventilator’ engine to work according to such individualized parameters.

As abovementioned, in order to facilitate the system to work with different parameters for each of the connected patients, the ventilator settings need to be determined in such a way that the ventilator itself can work with individual parameters for each patient and the medical personnel can select those different personal parameters for each patient on the screen display. The most important individual patients' parameters that must be individualized in terms of both ventilator activity and on-screen display of the ventilator:

1. Mode of operation—the mode in which the ventilator will work for each patient. Several basic ventilator modes can be used in modern day ventilators. The basic ventilator modes are: Continuous Mandatory Ventilation (CMV), where the ventilator is set to give a minimum number of guaranteed breaths every minute, Assist/Control ventilation (A/C), Pressure Support Ventilation (PSV), and Synchronized Intermittent Mandatory Ventilation (SIMV) with PS, a hybrid mode of the first two, continuous positive airway pressure (CPAP)).

2. Whether the patient receives the oxygen through the non-invasive mechanical ventilation (mask placed on his mouth or nose) or by invasive ventilation (inserted through a surgical opening, via tracheostomy, wherein a tube may need to be placed through an opening in the neck).

3. The amount of oxygen per patient.

4. The oxygen pressure per patient.

5. Frequency (f) or Respiratory Rate (RR) (normally 10-20 breaths/min) for each patient.

6. The exhaled oxygen quantity (to determine whether the medical personnel will increase or decrease the amount of oxygen)

7. Tidal Volume (Vt) Volume of gas exchanged with each breath.

8. The oxygen percentage in the inspired gas (FIO2).

9. Isens parameter. This parameter determines if the patient able to breathe on his own.

10. % Spontaneous Ventilation of each patient (Spont).

11. Percent Support.

12. Inspiratory Time.

Inspiratory time is defined as the period from the start of inspiratory flow to the start of expiratory flow. Inspiratory time has two components; inspiratory flow time (period when inspiratory flow is above zero) and inspiratory pause time (period when flow is zero).

13. Expiratory time is defined as the period from the start of expiratory flow to the start of inspiratory flow. The expiratory time is commonly dependent on the set inspiratory time, and set respiratory rate.

14. I:E Ratio. I:E is the ratio of inspiratory time to expiratory time.

15. I/T ratio.

16. Positive Expiratory End Pressure (PEEP). The PEEP is established by the ventilator exhalation valve.

17. Peak inspiratory flow. Its application is limited to volume control and volume assist breaths with a constant inspiratory flow.

18. Flow cycle is a cycling mechanism based on the inspiratory flow.

Setting a personal alarm for each patient.

Ventilator alarms bring unsafe events to the attention of the medical personnel. Such events can be classified according to their level of priority/severity.

Immediately life-threatening events are classified as Level 1 (alarm cannot be turned off). They include conditions like insufficient or excessive gas delivery to the patient or loss of power.

Level 2 events can be small irregularities in ventilator function or dangerous (but not immediately life-threatening) situations. Alarms in this category may be turned off.

In addition, and similarly to personalized parameters for each patient, personalized alarms must be prepared and produced. The most important alarms are:

1. Apnea alarms—a patient breathes less than a minimum or stops breathing on his own.

2. Inspiration trigger.

3.MinPip personality alarm for each patient.

3. PEEP High/PEEP Low alarms.

4. Too much oxygen for a patient.

5. Oxygen leak.

6. Alarm due to over-exhalation and/or over-breathing of high-vte air.

7. Alarm due to exhalation/respiration time index i/e ratio+i/t ratio is too high

### Display Device Means

The invented system further includes display device means 104, integrated in a wireless network, comprising an interface to enable medical professionals to calibrate ventilation parameters for all and each patient connected (105, 107). Such interface must include separate windows—one for each patient—to enable parameters personification for each patient. Such interfaces will be used in connection with the abovementioned programming means.

### Control System

To facilitate the processes of digitally controlling and commanding the multi-ventilator to work according to personalized patients' parameters and personalized patients' alarms, a control system must be included in the invented ventilator. Such a system (graphically depicted as 202 for patient 1 and 203 for patient 2) being connected to the abovementioned programming executable instructions means, and facilitates the commands provided by the abovementioned programming executable instructions means.

The control system comprises individualized electronic control circuits with microprocessors manage the monitoring and control functions of a ventilator. Such individualized electronic control circuits will be created for every patient connected to the ventilator. E.g., if five patients will be connected to a single multi-ventilator, then the said multi-ventilator will comprise five control circuits.

First, the system uses a microprocessor to control the gas delivery according to the preset parameters (such as volume, inspiratory time, flow pattern, etc.). In other words, microprocessors are used in the invented device and system for digital control of flow valves that allow a flexibility in shaping the ventilator's output pressure, volume, and flow waveforms.

The main type of controllers that must be individualized for every connected patient are: pressure controller for controlling air pressure parameters, volume controller for controlling air volume parameters, and flow controller for controlling flow parameters.

Generally, ventilators use closed-loop control to maintain consistent pressure and flow waveforms in the face of changing patient/system conditions, wherein feedback signal is used to adjust the output of a system, through using the output as a feedback signal that is compared to the pre-set input. The difference between the two is used to drive the system toward the desired output. The controller converts the error signal into a signal that drives the effector (the hardware) to cause a change in the manipulated ventilation variable.

From the abovementioned control hardware system 203 for patient 1 and 204 for patient 2, the individualized ventilation parameters get to each connected patient 206, 207.

### Database

The invented system further includes a database 108 for storing and analyzing the data obtained through ventilation.

The system database 301 collects data for 3 different databases as graphically described in FIG. 2. The database collects data for analysis and analysis on the inner disk.

1. The first database 302 collects safety data on all discharged and currently connected patients, including alert parameters 303 and history of alerts 304.

2. The second database 305 collects data on each patient's respiratory history.

The second database includes: patient general medical information 306, patient ventilator parameters 307, the history of alerts for each patient with the type and time of the alarm 308, and general medical procedures data 309 (not necessarily related to ventilation).

3. The third database 310 collects data on medical personnel (doctors, nurses and technicians) being employed currently or in the past in the ventilation process.

### Patients Ventilation Tubing and Separation Facilities

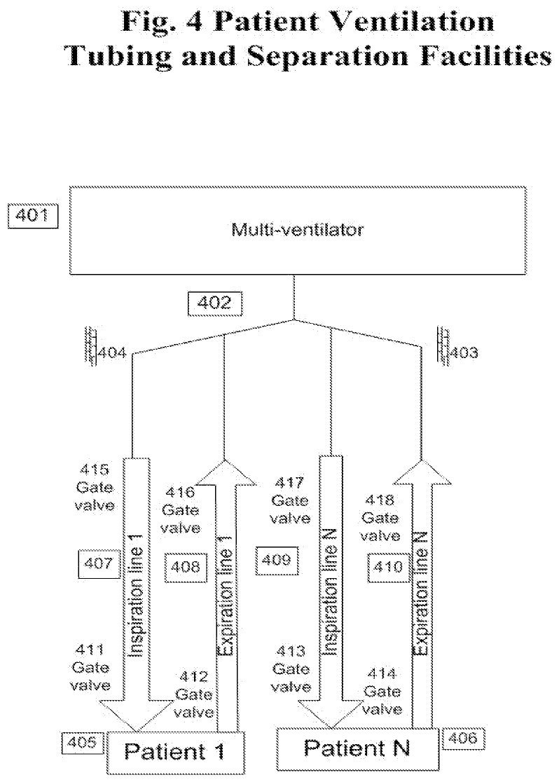

This paragraph is graphically presented in FIG. 4 for multi-ventilator 401.

To measure and to monitor a patient condition a flow sensor will be further included in expiration tubing of each patient, such sensors will be situated in an expiration tubing.

The flow sensor measures the actual flow, pressure and volume to allow for better assessment of the patients' condition. Patient condition will then be displayed on a patient monitor display.

Furthermore, inspiratory volume divider 402 is included to divide a total gas volume to each connected patient, comprising an individual inspiratory line for each connected patient, the said volume divider at one end having inspiratory branches for each patient; the abovementioned divider having measurement sensors (403 and 404) in each inspiratory branch for measuring a volume and a pressure of the gas delivered to each patient.

Separate inspiration and expiration lines will connect each connected patient (405, 406) to the ventilator. Inspiration and expiration lines controlled by the abovementioned central processor device to provide individualized ventilation for each connected patient. Such inspiration and expiration lines (407-410) must be separate so that bacteria and viruses are not transmitted from one patient to another.

Such tubing lines will have one-way inspiratory valves (411-414) and expiratory valves (415-418) for each connected patient at their ends to prevent mixing of gases.

Equipment used for ventilators is considered critical; such equipment should be cleaned and then receive high-level disinfection between patients. High-level disinfection of respiratory equipment takes place after cleaning, and is typically accomplished by chemical germicides or by hot-water pasteurization or by steam disinfection.

Thus, disinfection of ventilators is time-consuming and effort-consuming, as is the separation of the waste that comes out after the patient's breathing (saliva fragments, etc.). For example, pasteurization requires equipment to be submerged for at least 30 minutes in water at a temperature of about 70° C.

Using separate inspiration and expiration tubing lines in the invented ventilator system makes the ventilator extremely easy to clean and disinfect. It is also possible to make such tubing lines disposable.

Using separate inspiration and expiration tubing lines also enables easy separation of the waste that comes out after the patient's breathing.

### The Remote Wireless Operator Control Means

Current ventilators allow graphical displays of alarms, settings, respiratory system calculations, and measurements. Data are most commonly represented as numeric values such as FIO2, peak, plateau, mean and baseline airway pressures, inhaled/exhaled tidal volume, minute ventilation, and frequency.

The operator interaction with the invented multi-ventilator mainly happens through the ventilator display, called the operator interface.

The invented system further includes a Wireless Internet communication system to allow medical personnel to connect and to control the invented ventilator remotely and wirelessly. The central processor device 501 is connected to such a communication system. Such a Wireless Internet communication system 502 could be facilitated using the usual Wi-Fi Internet communication protocol. The Remote Wireless Operator Control Means 503 are enabled and integrated in such Wireless Internet communication system.

The mentioned operator interface includes one ventilator interface for each connected patient, wherein all individual patient display interfaces 504-506 are combined and are visible to the ventilator operators (medical personnel) from one single display. This allows the operator to scroll through different menus for each patient, to select and activate the individualized patient selections, and to customize the individualized screens according to individualized medical needs 508, 510, 512.

Using such remote monitoring feature eliminates the need of medical personnel to be physically near the infectious or potentially infectious patients. In such a way, the personnel will be protected from unnecessary exposure to viruses and bacteria. Additionally, it eliminates the need to wear special protective costumes, thus saving personnel' time and effort.

The operator interface, in addition to numerical values, provides individualized trend graphs of all parameters showing individualized trends of each patient.

Invented multi-ventilator operator interface further includes an individualized patient alarm, thus enabling the operator to view individual alarms for each patient.

Such individualized patient alarm log provides operator with access to date, time, alarm type, urgency level, and events associated with alarms.

Using such individualized operator interface together with individual alarms log is, also, important in terms of compliance, possible investigations, etc.

The described operator interface of the invention can be facilitated using a software package called LabView. LabView allows to design custom engineering user interfaces, and it offers a graphical programming approach that visualizes hardware configuration, measurement data, and debugging. This visualization makes it simple to integrate measurement hardware and presents data on graphs and diagrams. LabView can also help to develop ventilation data analysis algorithms.

The invented system further includes a remote patient visual monitoring feature on the abovementioned on-screen patient monitor display device, comprising an internet-connected web camera facing each patient 507, 509, 511 connected to the ventilator for visually monitoring the patient, for sending visual images of the patient to the said display device and for viewing these visual images of the patient on the said display device, such camera being integrated into the abovementioned wireless Internet communication system.

### Emergency Backup Systems

Because failure may result in death, ventilation systems are classified as life-critical systems, and precautions must be taken to ensure (or at least to minimize) that no failure can endanger the patient.

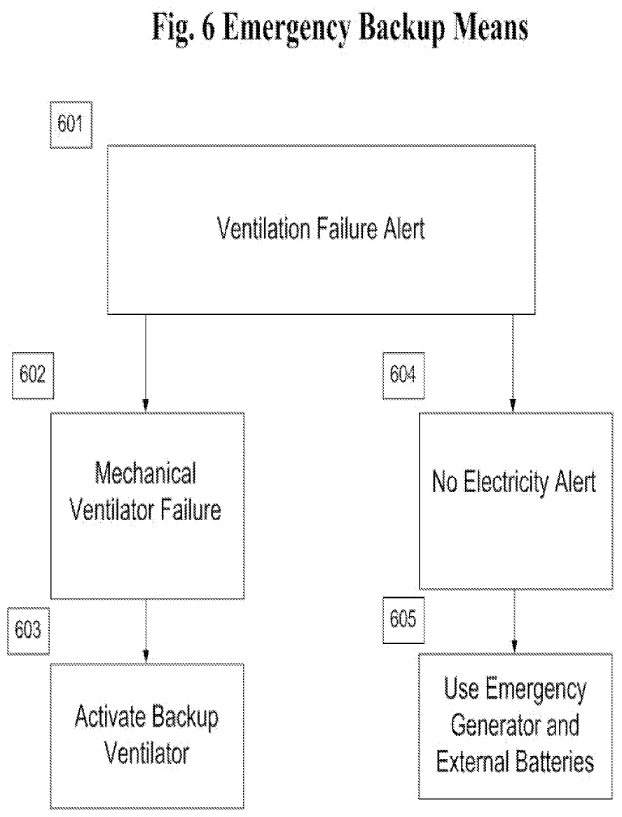

FIG. 6 graphically depicts emergency backup means (601) of the invented ventilator.

For a case of mechanical failure of the invented multi-ventilator (602), the invented system is further comprising backup multi-ventilator (603) identical to the multi-ventilator, such backup multi-ventilator would be configured with the same individualized ventilation care parameters for each connected patient as the original multi-ventilator.

Such backup multi-ventilator will be turned on and activated upon activation of a failure alarm for the original multi-ventilator, and thus will provide the same ventilation care to all connected patients in case of ventilator failure of the original multi-ventilator.

Such backup ventilator would be installed close to the working ventilator to immediately replace the broken ventilator in case of a failure.

In addition, hospital electrical power failure represents an important challenge in ventilators care. Total electrical power failure may occur (604). Therefore, a presence of backup generators is necessary. Additionally, a certain time period is required for rebooting the ventilator after an electric power outage has occurred.

Therefore, the invented ventilator must include external or internal backup batteries systems, in addition to the backup generators (605), in case that no electricity alarm is raised.

Battery backup systems are designed to continue operation for up to several hours after a power failure. There are a number of internal and external battery systems currently available in the market. Lifespan of currently available external batteries systems is much shorter than those of the external batteries systems. Examples of external batteries options are the Evita XL and Avea battery systems for ventilators. Those battery systems are known to have a lifespan of around 2-3 hours. For example, Evita XL external battery lifespan duration is around 3 hours.

Therefore, in the invented ventilator, the external battery system must be used.

What is claimed is:

1. A novel multi-ventilator system for treating multiple patients using one ventilator device, the system comprising:
a single ventilator device connected to a number of patients by inspiration and expiration tubing lines for simultaneously providing individualized different artificial ventilation care for two or more or more patients connected to the single ventilator device, the individualized ventilation different artificial ventilation care being provided differently to each patient according to patient-specific different ventilation care parameters, the patient-specific different ventilation care parameters being determined by medical personnel;
central processor device for controlling individualized ventilation care parameters of each connected patient to provide the individualized ventilation care, the central processor device further including a database for storing and analyzing the data obtained through ventilation of the connected patients;
a number of separate inspiration and expiration tubing lines controlled by the abovementioned central processor device, wherein each such tubing line will be connected to another connected patient, the tubing lines having one-way inspiratory and expiratory valves for each connected patient at their ends, the expiration tubing lines further having a flow sensor for monitoring of patient medical condition;
an inspiratory volume divider to divide a total gas volume to each patient connected, the inspiratory volume divider comprising an individual inspiratory tubing line for each connected patient, the volume divider at one end having inspiratory branches for
each patient; the inspiratory volume divider having measurement sensor in each inspiratory branch for measuring a volume and a pressure of the gas delivered to each patient;
a programming executable instructions to be connected with the central processor device, and also to be connected with the individual inspiratory and expiratory tubing lines, the programming executable instructions to enable the ventilator to work with the individualized parameters by sending programming commands to the ventilator engine, and thus, causing it to work according to such entered individualized parameters;
a control system for digitally controlling and commanding the ventilator device to work according to the personalized patients parameters and personalized patients alarms, such a system connected and facilitates the commands provided by the abovementioned programming executable instructions, the control system further comprising an individualized electronic control circuit with microprocessors for each patient connected to the ventilator device;
a wireless Internet communication system configured to allow medical personnel to connect and to control the ventilator remotely and wirelessly, the wireless internet communication system further includes connecting the ventilator device with an Internet connectivity, including Wi-Fi reception and communication, to allow the ventilator to be remotely connected and controlled;
a remote on-screen patient monitor display station, the remote station being connected with the programming executable instructions, and also being wirelessly connected through the wireless Internet communication to the ventilator and to the central processing device, for controlling the ventilator, for choosing different personal parameters for each connected patient and for displaying patient personalized ventilation abnormalities alarms; such remote station comprising an interface for entering and calibrating ventilation parameters for each patient connected, wherein the said interface includes a number of separate windows—one for each patient—to enable parameters personification for each connected patient.

2. The system of claim 1, further comprising a patient remote visual monitoring feature on the on-screen patient monitor display device, the patient remote visual monitoring feature comprising an internet-connected camera facing each patient connected to the ventilator for remotely visually monitoring the patient, for sending visual images of the patient to the said display device and for viewing these visual images of the patient on the said display device, the camera being integrated into the wireless Internet communication system of claim 1.

3. The system of claim 1, further comprising a backup multi-ventilator identical to the multi-ventilator of claim 1, the backup multi-ventilator configured with the same individualized ventilation care parameters for each connected patient as the said multi-ventilator of claim 1, the backup multi-ventilator will be turned on and activated upon activation of a failure alarm for the said multi-ventilator of claim 1, and thus will provide the same ventilation care to all connected patients in case of ventilator failure of the said multi-ventilator of claim 1.

4. The system of claim 1, further comprising external batteries and backup generators connected to the multi-ventilator to provide ventilation in case of an electric power failure alarm, the external batteries and backup generators configured to supply ventilation for all patient connected to the multi-ventilator system of claim 1.

5. The system of claim 1, wherein the personalized patient alarm shows a patient identifier, and an urgency of the alarm.

6. The system of claim 1, wherein the information about the personalized patient alarm comprises a ventilation care measured abnormal physiological parameter value corresponding to a patient.

7. A novel multi-ventilator device for treating two or more patients using single ventilator device, such a device being connected to a number of patients by inspiration and expiration tubing lines for simultaneously providing individualized different artificial ventilation care for two or more or more patients connected to the single ventilator device, the individualized ventilation different artificial ventilation care being provided differently to each patient according to patient-specific different ventilation care parameters, the patient-specific different ventilation care parameters being determined by medical personnel, the device comprising:
a central processor device configured to control individualized ventilation care parameters of each connected patient to provide the individualized ventilation care, the central processor device further including a database for storing and analyzing the data obtained through ventilation of the connected patients;
a number of separate inspiration and expiration tubing lines controlled by the abovementioned central processor device, wherein each such tubing line will be connected to another connected patient, the tubing lines having one-way inspiratory and expiratory valves for each connected patient at their ends;
an inspiratory volume divider configured to divide a total gas volume to each patient connected, the inspiratory volume divider comprising an individual inspiratory line for each connected patient, the volume divider at one end having inspiratory branches for each patient; the divider having measurement sensor in each inspiratory branch for measuring a volume and a pressure of the gas delivered to each patient;
a programming executable instructions connected with the central processor device, and also with the individual inspiratory and expiratory tubing lines, such programming executable instructions enabling the ventilator to work with the individualized parameters by sending programming commands to the ventilator engine, and thus, causing it to work according to such entered individualized parameters;
a control system configured to digitally control and command the ventilator device to work according to the personalized patients parameters and personalized patients alarms, such a system connected and facilitates the commands provided by the abovementioned programming executable instructions, the control system further comprising an individualized electronic control circuit with microprocessors for each patient connected to the ventilator device;
a wireless Internet communication connectivity to allow medical personnel to connect and to control the ventilator remotely and wirelessly, and further including equipping the ventilator device with an Internet connectivity capability, including Wi-Fi reception and communication capability, to allow the ventilator to be remotely connected and controlled;
a remote on-screen patient monitor display station configured to be connected with the programming means, and also being wirelessly connected to the ventilator/central system, for controlling it and for choosing different personal parameters for each connected patient and for displaying patient personalized ventilation abnormalities alarms; such remote station comprising an interface for entering and calibrating ventilation parameters for each patient connected, wherein the said interface includes a number of separate windows—one for each patient—to enable parameters personification for each connected patient.

8. The device of claim 7, further comprising a remote patient visual monitoring feature on the on-screen patient monitor display device, comprising an internet-connected camera facing each patient connected to the ventilator for visually monitoring the patient, for sending visual images of the patient to the said display device and for viewing these visual images of the patient on the said display device, the camera being integrated into the wireless Internet communication system of claim 5.
